(19) United States
(12) Patent Application Publication
(10) Pub. No.: US 2026/0276612 A1
(43) Pub. Date: Sep. 17, 2026

(54) GAS SENSOR AND GAS SENSOR ARRAY COMPRISING THE SAME

(71) Applicants: AJOU UNIVERSITY INDUSTRY-ACADEMIC COOPERATION FOUNDATION, Suwon-si, Gyeonggi-do (KR); INDUSTRY-UNIVERSITY COOPERATION FOUNDATION HANYANG UNIVERSITY ERICA CAMPUS, Ansan-si, Gyeonggi-do (KR)

(72) Inventors: Sang Woon LEE, Suwon-si (KR); Tae Joo PARK, Ansan-si (KR); Se Eun KIM, Gunpo-si (KR); Ju Young SUNG, Suwon-si (KR); Ji Hyeon CHOI, Ansan-si (KR)

(73) Assignees: AJOU UNIVERSITY INDUSTRY-ACADEMIC COOPERATION FOUNDATION, Suwon-si, Gyeonggi-do (KR); INDUSTRY-UNIVERSITY COOPERATION FOUNDATION HANYANG UNIVERSITY ERICA CAMPUS, Ansan-si, Gyeonggi-do (KR)

(21) Appl. No.: 18/727,142

(22) PCT Filed: Mar. 26, 2024
(86) PCT No.: PCT/KR2024/003760

§ 371 Date: Apr. 29, 2026

(30) Foreign Application Priority Data
Apr. 3, 2023 (KR) 10-2023-0043292
Mar. 21, 2024 (KR) 10-2024-0038885

(51) Int. Cl. G01N 33/00

(52) U.S. Cl. CPC G01N 33/005; G01N 33/0031

(57) ABSTRACT

The present disclosure relates to a gas sensor including a first oxide layer, a second oxide layer disposed on the first oxide layer, an inactive electrode formed at an interface of the first oxide layer and the second oxide layer, and an active electrode disposed on the second oxide layer, the inactive electrode includes a two-dimensional electron gas (2DEG) layer, and a metal filament which connects the active electrode and the inactive electrode and is derived from the active electrode is included in the second oxide layer.

### TECHNICAL FIELD

The present disclosure relates to a gas sensor which detects gas within a short time of several nanoseconds or shorter and a gas sensor array including the same.

### BACKGROUND ART

Hydrogen is a sustainable energy source and is highly efficient without producing harmful materials, so that it is attracting attention in many countries as an important future energy source that can replace fossil energy sources for carbon neutrality.

In contrast, hydrogen is a material which is very small in size and has strong penetrating power to easily form pin holes or cracks to be easily exposed. Therefore, when a very small amount (ppm level) of hydrogen leaks due to the formed crack, the risk of explosion may increase due to flammability of hydrogen so that a gas sensing technology which can precisely detect hydrogen gas leakage is essential.

However, gas sensors developed to date to detect hydrogen require high operation temperature of 150° C. to 400° C. and require a detection time of several to tens of seconds so that there are still limitations in detecting hydrogen in the early stage of hydrogen leakage.

Accordingly, there is a need to develop an ultra-fast gas sensor which can detect gas within a short time of nano second level.

Korean Unexamined Patent Application Publication No. 10-2021-0050282 which is a background art of the present disclosure relates to a GaN based semiconductor gas detector combined heater and a method for manufacturing the same and specifically, provides a GaN based semiconductor gas detector combined heater in which a semiconductor gas sensor which reacts gas to be detected and a GaN based semiconductor heating device are disposed to improve the reactivity of the semiconductor gas sensor.

### DETAILED DESCRIPTION OF THE INVENTION

### Technical Problem

The present disclosure is provided to solve the above-described problems of the related art and provides a gas sensor which is capable of detecting gas within a short time of nano second level.

Further, the present disclosure provides a gas sensor array including the gas sensor.

However, objects to be achieved by the embodiments of the present disclosure are not limited to the technical objects as described above and other technical objects may be present.

### Technical Solution

As a technical means to achieve the above-described technical object, according to a first aspect of the present disclosure, a gas sensor includes a first oxide layer; a second oxide layer disposed on the first oxide layer; an inactive electrode formed at an interface of the first oxide layer and the second oxide layer; and an active electrode disposed on the second oxide layer, the inactive electrode includes a two-dimensional electron gas (2DEG) layer, and a metal filament which connects the active electrode and the inactive electrode and is derived from the active electrode is included in the second oxide layer.

According to an implementation embodiment of the present disclosure, when gas is injected in a state in which a voltage is applied to the gas sensor, the connection of the metal filament and the inactive electrode is disconnected and when the injection of the gas is stopped, the metal filament and the inactive electrode are connected again, but is not limited thereto.

According to an implementation embodiment of the present disclosure, when the gas is injected, due to the heat transmission by the gas, a temperature of a part of the metal filament adjacent to the inactive electrode exceeds a critical point so that the metal filament collapses, but is not limited thereto.

According to an implementation embodiment of the present disclosure, in the collapse state, a tunnel gap which is an interval between an end portion of the metal filament and the inactive electrode is 0.1 nm or larger and 0.5 nm or smaller, but is not limited thereto.

According to an implementation embodiment of the present disclosure, a diameter of a part of the metal filament adjacent to the active electrode is larger than a diameter of a part of the metal filament adjacent to the inactive electrode, but is not limited thereto.

According to an implementation embodiment of the present disclosure, the gas includes one selected from the group consisting of hydrogen, ammonia, benzene, butane, heptane, hexane, pentane, propylene, isobutane, ethylene, ethane, isopentane, isobutylene, methane, propane, hydrogen sulfide, carbon monoxide, and combinations thereof, but is not limited thereto.

According to an implementation embodiment of the present disclosure, the gas sensor further includes a buffer layer disposed between the active electrode and the second oxide layer, but is not limited thereto.

According to an implementation embodiment of the present disclosure, the first oxide layer and the second oxide layer are each independently formed of oxide including a metal element selected from the group consisting of titanium (Ti), zinc (Zn), aluminum (Al), hafnium (Hf), zirconium (Zr), gallium (Ga), tungsten (W), indium (In) and combinations thereof, but is not limited thereto.

According to an implementation embodiment of the present disclosure, the active electrode includes one selected from the group consisting of copper (Cu), silver (Ag), platinum (Pt), gold (Au), molybdenum (Mo), nickel (Ni), and combinations thereof, but is not limited thereto.

Further, according to a second aspect of the present disclosure, a gas sensor array includes a plurality of gas sensors which is disposed in a M×N matrix along a first horizontal direction and a second horizontal direction intersecting the first horizontal direction; a plurality of first signal lines which is disposed in the second horizontal direction to be spaced from each other and extends to the first horizontal direction to be electrically connected to active electrodes of M gas sensors 10 disposed in one line along the first horizontal direction, among the plurality of gas sensors, and a plurality of second signal lines which is disposed in the first horizontal direction to be spaced from each other and extends to the second horizontal direction to be electrically connected to inactive electrodes of N gas sensors disposed in one line along the second horizontal direction, among the plurality of gas sensors, the plurality of gas sensors includes a gas sensor according to the first aspect of the present disclosure and the plurality of gas sensors has different thicknesses of parts of the metal filament which are in contact with the inactive electrode.

According to an implementation embodiment of the present disclosure, when the gas is injected, due to the heat transmission by the gas, a temperature of a part of the metal filament adjacent to the inactive electrode exceeds a critical point so that the metal filament collapses and the collapse selectively occurs according to a thickness of the metal filament and a concentration of the injected gas, but is not limited thereto.

According to an implementation embodiment of the present disclosure, each of the plurality of gas sensors further includes a support structure which is disposed on one corresponding second signal line, among the second signal lines and has a pillar structure, the first oxide layer is disposed to cover a side surface and an upper surface of the support structure, and the second oxide layer is disposed to directly cover the first oxide layer on the side surface and the upper surface of the support structure, but is not limited thereto.

According to an implementation embodiment of the present disclosure, the support structure includes a pillar structure having a circular or polygonal cross-sectional shape, but is not limited thereto.

According to an implementation embodiment of the present disclosure, the second signal lines are in direct contact with cross-sectional surfaces of the inactive electrodes exposed in an area adjacent to a lower end portion of the support structure, but are not limited thereto.

According to an implementation embodiment of the present disclosure, the gas sensor array further includes a filling layer disposed to fill a space between the plurality of gas sensors, and active electrodes of the gas sensors are exposed through an upper surface of the filling layer to be electrically connected to the first signal line, but are not limited thereto.

According to an implementation embodiment of the present disclosure, the filling layer includes a first layer which fills a space between the gas sensors and a second layer formed above the first layer, the first layer exposes a part of an upper surface of the second oxide layer of the gas sensors, and the second layer includes a plurality of through-holes through which the active electrodes of the gas sensors pass, but is not limited thereto.

The above-described solving means are merely illustrative but should not be construed as limiting the present disclosure. In addition to the above-described embodiments, additional embodiments may be further provided in the drawings and the detailed description of the present disclosure.

### Effects of the Invention

The gas sensor according to the present disclosure includes a metal filament which connects an active electrode and an inactive electrode including a two-dimensional electron gas layer. When gas is injected in a state in which the voltage is applied to the gas sensor, a part of the metal filament adjacent to the inactive electrode collapses so that the connection between the active electrode and the inactive electrode is disconnected, which causes the current to be rapidly reduced. Therefore, the gas may be detected at an ultra-high speed at a nano second level.

Further, in the gas sensor of the related art, the recovery time is very long to be several minutes, but in the gas sensor according to the present disclosure, even though gas injection is stopped, a voltage of several volts is maintained in the metal filament, so that the ultra-high speed recovery is possible within several nano seconds.

Further, the gas sensor according to the present disclosure is manufactured by adjusting a size of the metal filament so that the gas is detected by setting a size of the metal filament which reacts an optimal concentration of gas to more efficiently sense the gas.

A gas sensor array according to the present disclosure including a plurality of gas sensors according to the present disclosure has metal filaments having different thicknesses, included in each gas sensor so that the metal filament selectively collapses according to the thickness of the metal filament and the concentration of gas to be injected when the gas is injected, which allows the measurement of the concentration of gas to be injected.

However, the effect which can be achieved by the present disclosure is not limited to the above-described effects, there may be another effects.

### BRIEF DESCRIPTION OF THE DRAWINGS

FIG. 1 is a diagram of a gas sensor according to one implementation embodiment of the present disclosure.

FIG. 2 is a cross-sectional view of a gas sensor array according to one implementation embodiment of the present disclosure.

FIG. 3 is a flowchart of a manufacturing process of a gas sensor array according to one embodiment of the present disclosure.

FIG. 4 is a diagram of an experiment of measuring reaction and recovery speed of a gas sensor according to one experimental embodiment of the present disclosure.

FIG. 5 is a result of measuring a reaction time when hydrogen is injected at 1% hydrogen concentration into a gas sensor according to embodiment 1-1 of the present disclosure.

FIG. 6 is a result of measuring a recovery time when injection of hydrogen at 1% hydrogen concentration is stopped into a gas sensor according to embodiment 1-1 of the present disclosure.

FIG. 7 is a results of measuring the reaction time and recovery time when hydrogen injection and hydrogen injection are stopped at 0.5% hydrogen concentration into the gas sensor according to embodiment 1-2 of the present disclosure.

FIG. 8 is a results of measuring the reaction time and recovery time when hydrogen injection and hydrogen injection are stopped at 0.1% hydrogen concentration into the gas sensor according to embodiment 1-3 of the present disclosure.

### BEST MODE FOR CARRYING OUT THE INVENTION

Hereinafter, the present disclosure will be described more fully hereinafter with reference to the accompanying drawings, in which exemplary embodiments of the present disclosure are shown. However, the present disclosure may be realized in various different forms, and is not limited to the embodiments described herein. Accordingly, in order to clearly explain the present disclosure in the drawings, portions not related to the description are omitted. Like reference numerals designate like elements throughout the specification.

Throughout this specification and the claims that follow, when it is described that an element is “coupled” to another element, the element may be “directly coupled” to the other element or “electrically coupled” to the other element through a third element.

Through the specification of the present disclosure, when one member is located “on”, “above”, “on an upper portion”, “below”, “under”, and “on a lower portion” of the other member, the member may be adjacent to the other member or a third member may be disposed between the above two members.

In the specification, unless explicitly described to the contrary, the word “comprise” and variations such as “comprises” or “comprising”, will be understood to imply the inclusion of stated elements but not the exclusion of any other elements.

The terms “about or approximately” or “substantially” indicating a degree used throughout the specification are used as a numerical value or a meaning close to the numerical value when a unique manufacturing and material tolerance is proposed to the mentioned meaning and also used to prevent unscrupulous infringers from wrongfully using the disclosure in which precise or absolute numerical values are mentioned for better understanding of the present disclosure. Terms used throughout the specification, “~step of ~ing” or “step of~” do not mean “step for~”.

Throughout the specification of the present disclosure, the term “combination thereof” included in the expression of Markushi format refers to a mixture or a combination of one or more selected from the group consisting of components described in the expression of the Markushi format and it means that one or more selected from the group consisting of the components is included.

Throughout the specification of the present disclosure, the description of “A and/or B” refers to “A, B or A and B”.

Hereinafter, a gas sensor according to the present disclosure and a gas sensor array including the same will be described in detail with reference to an implementation embodiment, Embodiments, and drawings. However, the present disclosure is not limited to the implementation embodiment, Embodiments, and the drawings.

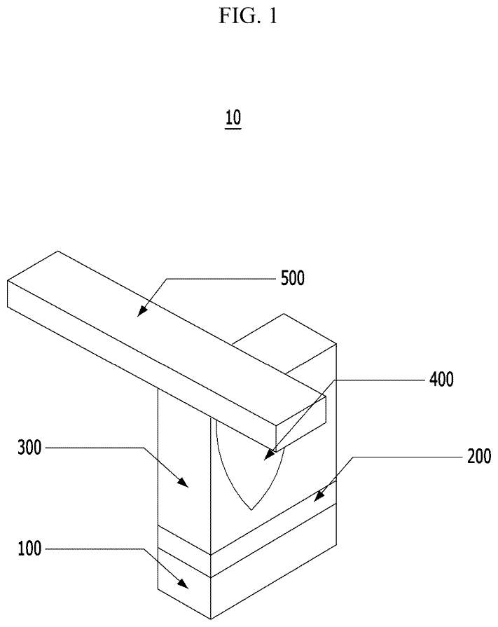

FIG. 1 is a diagram of a gas sensor 10 according to one implementation embodiment of the present disclosure.

As a technical means for achieving the above-described technical object, a first aspect of the present disclosure provides a gas sensor 10 including a first oxide layer 100, a second oxide layer 300 disposed on the first oxide layer 100, an inactive electrode 200 formed on an interface of the first oxide layer 100 and the second oxide layer 300, and an active electrode 500 disposed on the second oxide layer 300. The inactive electrode 200 includes a two-dimensional electron gas (2DEG) layer and the active electrode 500 and the inactive electrode 200 are connected into the second oxide layer 300 and a metal filament 400 derived from the active electrode 500 is included.

A heat conduction based sensor of the related art measures a phenomenon of decrease of current which flows due to the increase of temperature of metal when the gas is injected, and is advantageous for sensing because the smaller the volume and the larger the surface area of the sensing material which is formed of metal, the larger the temperature change by the gas injection. Accordingly, in order to reduce the volume of the metal which is a sensing material, it is manufactured as a thin wire and is manufactured in a size of several micrometers using MEMS technology. Generally, the thin wire is used to be floated in the air to increase the sensitivity of the sensor.

If a metal wire is manufactured to have a stable form with a size of several nanometers, it may be developed as an ultra-fast and ultra-high sensitivity sensor device. However, when the metal wire is manufactured to have a nanometer size which is much smaller than the micrometer scale, expensive EUV photolithography process needs to be used, rather than a general photolithography, and a thickness of the formed wire is very thin so that there are problems in that it is difficult to maintain a floating state in the air and the wire is easily broken due to the external shock.

However, the gas sensor 10 according to the present disclosure uses a metal filament 400 having a stable form with several nanometer size as a sensing device so that it is possible to provide an ultra-fast and ultra-high sensitivity gas sensor.

Specifically, when a voltage is applied to the gas sensor 10, an active metal ion is injected into the second oxide layer 300 from the active electrode 500 to move toward the inactive electrode 200 and is reduced around the inactive electrode 200 to form the metal filament 400 in the second oxide layer 300 to electrically connect the active electrode 500 and the inactive electrode 200.

A two-dimensional electron gas layer formed by binding high concentration of electrons (10¹² to 1014/cm²) is included at the interface of the first oxide layer 100 and the second oxide layer 300 and is used as a metal electrode. Accordingly, the gas sensor 10 according to the present disclosure uses the two-dimensional electron gas layer present at the interface of the first oxide layer 100 and the second oxide layer 300 as the inactive electrode 200, instead of a counter electrode of the related art, such as Pt or TiN and forms the metal filament 400 which connects the active electrode 500 and the inactive electrode 200 to be used as a sensing device to provide a ultra-fast and ultra-high sensitivity gas sensor.

The electrons of the two-dimensional electron gas layer are restricted from moving out of the interface, but freely move along the interface, thereby exhibiting electrical conductivity characteristic.

In the meantime, the gas sensor 10 according to the present disclosure is manufactured by adjusting a size of the metal filament 400 so that the gas is detected by setting a size of the metal filament 400 which reacts an optimal concentration of gas to more efficiently sense the gas.

According to one implementation embodiment of the present disclosure, when the gas is injected in a state in which the voltage is applied to the gas sensor 10, the connection between the metal filament 400 and the inactive electrode 200 is disconnected and when the injection of the gas is stopped, the metal filament 400 and the inactive electrode 200 are connected again, but it is not limited thereto.

When the voltage is applied to the gas sensor 10, the metal filament 400 which electrically connects the active electrode 500 and the inactive electrode 200 is formed and when the gas is injected to the gas sensor 10 in a state in which the voltage is applied, the connection between the metal filament 400 and the inactive electrode 200 is disconnected, which causes the current to rapidly decrease.

According to one implementation embodiment of the present disclosure, when the gas is injected, due to the heat conduction by the gas, a temperature of a part of the metal filament 400 adjacent to the inactive electrode 200 exceeds a critical point and the metal filament collapses, but is not limited thereto.

When a voltage is applied to the metal filament 400, the temperature of the metal filament 400 is increased by joule heating and at this time, the temperature exceeds the critical point, the metal filament 400 may collapse.

Accordingly, an appropriate voltage is applied to maintain a temperature of the metal filament 400 to be equal to or lower than the critical point. When the gas is injected in this state, the temperature of the metal filament 400 exceeds the critical point due to the heat conduction by the gas and thus, a part of the metal filament 400 adjacent to the inactive electrode 200 collapses so that the connection between the active electrode 500 and the inactive electrode 200 is disconnected, which may cause the current to rapidly decrease.

In the meantime, when the gas injection is stopped, the metal filament 400 which collapses by the gas injection may recover a state in which the active electrode 500 and the inactive electrode 200 are connected within a short time of several nanoseconds.

According to one implementation embodiment of the present disclosure, in the collapsing state, a tunnel gap which is an interval between an end portion of the metal filament 400 and the inactive electrode 200 may be 0.1 nm or larger and 0.5 nm or smaller, but is not limited thereto.

The gas sensor 10 according to the present disclosure uses the inactive electrode 200 including the two-dimensional electron gas layer so that a relatively small metal filament 400 may be formed in the second oxide layer 300. Further, an extremely short tunnel gap is formed during the collapse, so that the control over the formation and the destruction of the metal filament 400 may be improved.

According to one implementation embodiment of the present disclosure, a diameter of a part of the metal filament 400 adjacent to the active electrode 500 is larger than a diameter of a part of the metal filament 400 adjacent to the inactive electrode 200, but is not limited thereto.

In order to form the size of the metal filament 400 to be small, a reduction rate of the active metal ions in the inactive electrode 200 needs to be sufficiently slow and a reduction rate of the active metal ions at the interface of the first oxide layer 100 and the second oxide layer 300 may be much slower than a reduction rate of noble metals such as Pt. The two-dimensional electron gas included in the inactive electrode 200 exhibits a semi-metallic electrical conductivity so that an ability to supply electrons to the active metal ions may be lower than that of the noble metal Pt. Therefore, in the case of the gas sensor 10 according to the present disclosure which applies the inactive electrode 200 including the two-dimensional electron gas layer, instead of the counter electrode of the related art, such as Pt or TiN, an extremely small metal filament 400 may be formed in the vicinity of the inactive electrode 200 and consequently, the gas sensing ability may be improved.

According to one implementation embodiment of the present disclosure, the first oxide layer 100 and the second oxide layer 300 are each independently formed of oxide including a metal element selected from the group consisting of titanium (Ti), zinc (Zn), aluminum (Al), hafnium (Hf), zirconium (Zr), gallium (Ga), tungsten (W), indium (In) and a combination thereof, but are not limited thereto.

For example, each of the first oxide layer 100 and the second oxide layer 300 may be formed of one metal oxide selected from titanium oxide (for example, TiO₂, SrTiO₃, etc.), zinc oxide (for example, ZnO, etc.), aluminum oxide (for example, Al₂O₃, LaAlO₃, etc.), and hafnium oxide (for example, HfO, etc.), zirconium oxide (for example, ZrO, etc.), gallium oxide (for example, Ga₂O₃, etc.), tungsten oxide (for example, WOₓ, etc.), and IGZO(InGaZnO). In this case, the first oxide layer 100 and the second oxide layer 300 may be formed of different materials. As one example, the second oxide layer 300 which is located between the active electrode and the inactive electrode 200 to have a conductive filament 400 formed therein may be formed of aluminum oxide or tungsten oxide, such as Al₂O₃, LaAlO₃, WOₓ, etc. The first oxide layer 100 may be formed of titanium oxide, such as TiO₂ and SrTiO₃.

In the meantime, when a thickness of the first oxide layer 100 and/or the second oxide layer 300 is thinner than approximately 2.5 nm, the two-dimensional electron gas layer may not be formed so that each of the first oxide layer 100 and the second oxide layer 300 may have a thickness of approximately 2.5 nm or larger. For example, each of the first oxide layer 100 and the second oxide layer 300 may formed to have a thickness of approximately 5 to 500 nm.

According to one implementation embodiment, the active electrode 500 may include one selected from the group consisting of copper (Cu), silver (Ag), platinum (Pt), gold (Au), molybdenum (Mo), nickel (Ni), and combinations thereof, but is not limited thereto.

The active electrode 500 may be disposed above the second oxide layer 300 and may supply active metal ions into the second oxide layer 300 under the condition that a positive bias voltage is applied. For example, if the active electrode 500 is copper (Cu), when a positive bias voltage is applied, copper (Cu) ions may be supplied into the second oxide layer 300.

According to one implementation embodiment of the present disclosure, a buffer layer disposed between the active electrode 500 and the second oxide layer 300 may further be included, but is not limited thereto.

The buffer layer may serve to suppress excessive active metal ions from moving from the active electrode 500 into the second oxide layer 300 under the condition that the positive bias voltage is applied, but is not limited thereto.

According to one implementation embodiment of the present disclosure, the gas may include one selected from the group consisting of hydrogen, ammonia, benzene, butane, heptane, hexane, pentane, propylene, isobutane, ethylene, ethane, isopentane, isobutylene, methane, propane, hydrogen sulfide, carbon monoxide, and combinations thereof, but is not limited thereto.

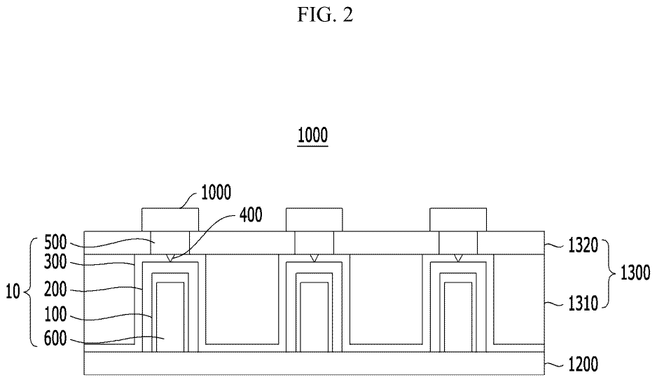

FIG. 2 is a cross-sectional view of a gas sensor array 1000 according to one implementation embodiment of the present disclosure.

A second aspect of the present disclosure provides a gas sensor array 1000 including a plurality of gas sensors 10 which is disposed in a M×N matrix along a first horizontal direction and a second horizontal direction intersecting the first horizontal direction, a plurality of first signal lines 1100 which is disposed in the second horizontal direction to be spaced from each other and extends to the first horizontal direction to be electrically connected to active electrodes 500 of M gas sensors 10 disposed in one line along the first horizontal direction, among the plurality of gas sensors 10, and a plurality of second signal lines 1200 which is disposed in the first horizontal direction to be spaced from each other and extends to the second horizontal direction to be electrically connected to inactive electrodes 200 of N gas sensors 10 disposed in one line along the second horizontal direction, among the plurality of gas sensors 10. The plurality of gas sensors 10 includes the gas sensors 10 according to the first aspect of the present disclosure and in the plurality of gas sensors 10, thicknesses of parts of the metal filaments 400 which are in contact with the inactive electrodes 200 are different.

A detailed description of repeated parts of the gas sensor array 1000 according to the second aspect of the present disclosure with the first aspect of the present disclosure will be omitted. However, even though the detailed description thereof is omitted, the description of the first aspect of the present disclosure may be applied to the second aspect of the present disclosure in the same manner.

According to one implementation embodiment of the present disclosure, when the gas is injected, due to the heat conduction by the gas, the temperature of a part of the metal filament 400 adjacent to the inactive electrode 200 exceeds a critical point and the metal filament collapses. The collapse selectively occurs according to the thickness of the metal filament 400 and a concentration of the injected gas, but is not limited thereto.

The gas sensor array 1000 according to the present disclosure includes a plurality of gas sensors 10 and the metal filaments 400 included in the plurality of gas sensors 10 may be formed to have different thicknesses. Accordingly, when the gas is injected, a part of the metal filament 400 adjacent to the inactive electrode 200 may selectively collapse according to the thickness of the metal filament 400 and a concentration of the injected gas.

For example, when heat is transmitted to the metal filaments 400 by the gas injection, the metal filament 400 having a relatively small thickness exceeds a critical point to collapse. In contrast, the metal filament 400 having a relatively large thickness does not exceed the critical point, so that the metal filament does not collapse.

As another example, when the gas concentration is low, the temperature does not reach the critical point due to the insufficiently transmitted heat so that the metal filament 400 does not collapse. However, due to the increase concentration of the injected gas, the transmitted heat increases so that the temperature exceeds the critical point and the metal filament may collapse.

Accordingly, the collapse of the metal filament 400 may selectively occur according to the thickness of the metal filament 400 and the concentration of the injected gas and the gas concentration is measured using the collapse characteristic of the metal filament 400, but is not limited thereto.

According to one implementation embodiment of the present disclosure, each of the plurality of gas sensors 10 further includes a support structure 600 which is disposed on a corresponding second signal line, among the second signal lines 1200 and has a pillar structure. The first oxide layer 100 is disposed to cover a side surface and an upper surface of the support structure 600 and the second oxide layer 300 is disposed to directly cover the first oxide layer 100 on the side surface and the upper surface of the support structure 600, but is not limited thereto.

The support structure 600 may be disposed on the second signal line 1200 and the first oxide layer may be disposed to cover the side surface and the upper surface of the support structure 600. For example, the first oxide layer 100 may be formed by forming a first oxide thin film with a predetermined thickness to cover the side surface and the upper surface of the support structure 600 by means of the ALD process and then patterning the first oxide thin film. Further, the second oxide layer 300 may be disposed to directly cover the first oxide layer 100 on the side surface and the upper surface of the support structure 600. For example, the second oxide layer 300 may be formed by forming the first oxide layer 100 and then forming the second oxide thin film thereon by means of the ALD process.

The higher the ALD process temperature, the higher the two-dimensional electron gas (2DEG) concentration. Accordingly, samples manufactured under high temperature conditions have a high 2DEG concentration, so the standby current level is high and can be advantageous for high concentration detection. Conversely, samples prepared under low temperature conditions have a low 2DEG concentration and therefore a low standby current level, which may be advantageous for low concentration detection.

According to one implementation embodiment, the support structure 600 may include a pillar structure having a circular or polygonal cross-sectional shape, but is not limited thereto.

The support structure 600 may have a pillar structure having a constant diameter, but may also have a pillar structure in which an upper surface and a lower surface have different diameters. For example, the support structure 600 may have a pillar structure having a circular or square cross-section and constant diameters. Alternatively, a diameter of the lower surface may be larger than a diameter of the upper surface. The support structure 600 may be formed of an electrical insulating material and a material is not specifically limited as long as the material has an insulating property.

As described above, when the first oxide layer 100 and the second oxide layer 300 are formed to have an interface, a two-dimensional electron gas layer may be formed at the interface and may be used as the inactive electrode 200.

The plurality of first signal lines 1100 may be disposed above the plurality of gas sensors 10 and may electrically be connected to the active electrodes 500 of the plurality of gas sensors 10.

As one embodiment, the plurality of first signal lines 1100 may be disposed to be spaced apart from each other in the second horizontal direction and each first signal line 1100 extends along the first horizontal direction. Each of the first signal lines 1100 is electrically connected to the active electrodes 500 of M gas sensors 10 which are disposed in one line along the first horizontal direction, among the plurality of gas sensors 10 and may apply a first voltage to the active electrodes 500 of the M gas sensors 10.

The plurality of second signal lines 1200 may be disposed below the plurality of gas sensors 10 and may electrically be connected to the inactive electrodes 200 of the plurality of gas sensors 10. For example, the support structures 600 of the gas sensors 10 are disposed on corresponding second signal lines 1200, respectively, to be supported thereby.

For example, the plurality of second signal lines 1200 is disposed to be spaced apart from each other in the first horizontal direction and each second signal line 1200 may extend along the second horizontal direction. Each of the second signal lines 1200 is electrically connected to the inactive electrodes 200 of N gas sensors 10 which are disposed in one line along the second horizontal direction, among the plurality of gas sensors 10 and may apply a second voltage to the inactive electrodes 200 of the N gas sensors 10. For example, the second signal lines 1200 are in direct contact with end surfaces of the inactive electrodes 200 exposed in a lower area of the support structure 600 to be electrically connected to the inactive electrodes 200.

An electric field is applied to the second oxide layer 300 by first and second voltages which are applied to the active electrode 500 and the inactive electrode 200 through the first signal line 1100 and the second signal line 1200, respectively. The metal filament 400 may be formed in the second oxide layer 300, for example, a part of the second oxide layer 300 which is located on the upper surface of the support structure 600 by the electric field.

According to one implementation embodiment of the present disclosure, a filling layer 1300 is further disposed to fill a space between the plurality of gas sensors 10 and the active electrodes 500 of the gas sensors 10 is exposed through the upper surface of the filling layer 1300 to be electrically connected to the first signal lines 1100, but is not limited thereto.

The gas sensor array 1000 according to the present disclosure may further include a filling layer 1300 which is formed of an electrical insulating material, to improve a structural stability.

According to one implementation embodiment of the present disclosure, the filling layer 1300 includes a first layer 1310 which fills a space between the gas sensors 10 and a second layer 1320 formed above the first layer 1310. The first layer 1310 exposes a part of the upper surfaces of the second oxide layers 300 of the gas sensors 10 and the second layer 1320 includes a plurality of through-holes through which the active electrodes 500 of the gas sensors 10 pass, but is not limited thereto.

Hereinafter, the present disclosure will be described in more detail with respect to Embodiments, but the following Embodiments are set forth to illustrate, but are not to be construed to limit the scope of the present disclosure.

### [Embodiment 1-1] Manufacture Gas Sensor

A TiO₂ film with a thickness of approximately 20 nm was formed on a substrate by means of an atomic layer deposition (ALD) process of 250° C. At this time, as sources of titanium and oxide of the TiO₂ film, titanium tetraisoproxide (Ti{OCH(CH₃)₂}₄, TTIP) and water (H₂O) were used.

Next, in order to produce two-dimensional electron gas (2DEG), an Al₂O₃ thin film with a thickness of approximately 7 nm was formed on the TiO₂ film by means of the ALD process of 300° C. At this time, as sources of aluminum and oxide of the Al₂O₃ thin film, trimethylaluminum (Al(CH₃)₃, TMA) and water (H₂O) were used.

Next, an active electrode which had a Ti (2 nm)/Cu (40 nm) laminated structure and was patterned to have a square shape was formed on the Al₂O₃ thin film by means of the E-beam deposition process and the photolithography process.

Next, an indium bottom contact which was in contact with the two-dimensional electron gas was formed by a soldering process to manufacture a gas sensor according to Embodiment.

### [Embodiment 1-2] Manufacture Gas Sensor

It was prepared in the same manner as Embodiment 1-1, but the 2DEG concentration was adjusted by changing the Al₂O₃ ALD process temperature to 275° C.

### [Embodiment 1-3] Manufacture Gas Sensor

It was prepared in the same manner as Embodiment 1-1, but the 2DEG concentration was adjusted by changing the Al₂O₃ ALD process temperature to 250° C.

### [Embodiment 2] Manufacture Gas Sensor Array

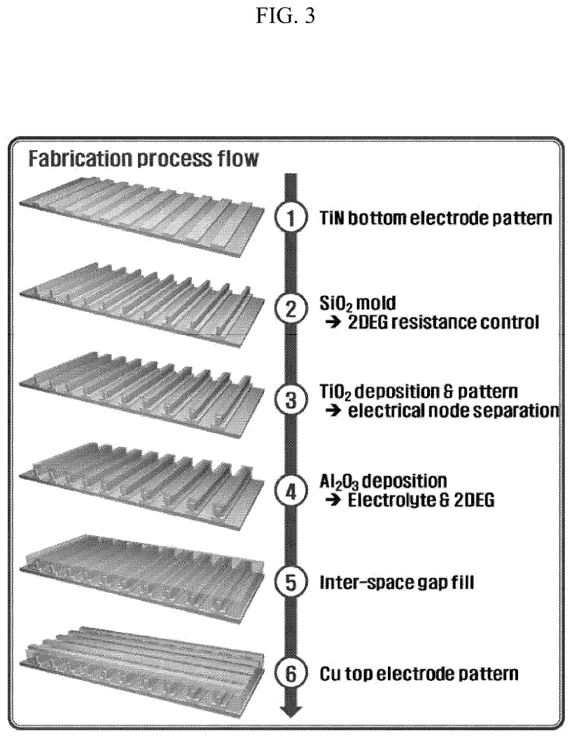

FIG. 3 is a flowchart of a manufacturing process of a gas sensor array according to one embodiment of the present disclosure.

Referring to FIG. 3, a TiN lower electrode was deposited by sputtering or the ALD process and patterning (dry etch) was conducted.

Next, SiO₂ was deposited on the TiN lower electrode by the ALD process and then a mold was formed by the dry etching process.

Next, a TiO₂ thin film was deposited on the SiO₂ by the ALD process and the patterning (dry etch) was conducted.

Next, an Al₂O₃ thin film was deposited on the TiO₂ thin film by the ALD process and the patterning (dry etch) was conducted.

Finally, a Ti (2 nm)/Cu (40 nm) top electrode was deposited on Al₂O₃ and patterning (dry etch) was conducted.

### [Experimental Embodiment 1] Detect Gas

An experiment for measuring reaction and recovery speed of the gas sensor manufactured by Embodiments 1-1 to 1-3 was conducted. Specifically, the gas sensors of Embodiments 1-1 to 1-3 were used to detect hydrogen concentrations of 1%, 0.5%, and 0.1%, respectively.

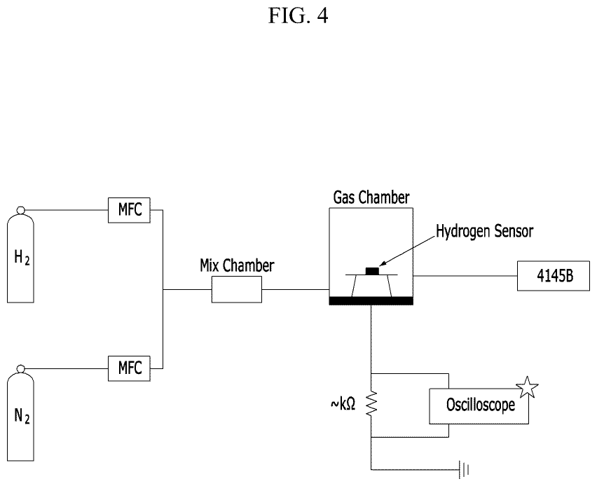

FIG. 4 is a diagram of an experiment of measuring reaction and recovery speed of a gas sensor according to one experimental embodiment of the present disclosure.

Referring to FIG. 4, the experiment was conducted after loading a sensor in a gas chamber and then making it an isolated system.

First, the hydrogen concentration was adjusted by injecting a mixture of H₂ 1% (N₂ 99%) gas and N₂ (100%) gas. Specifically, H₂ 1% gas was injected at 40 to 400 sccm through the MFC, and 400 sccm of N₂ 100% gas was also injected to adjust the hydrogen concentration to 0.1% to 1%.

Next, the gas sensors according to Embodiments 1-1 to 1-3 were placed in a chamber at room temperature, and connected to HP 4145 to measure the change in current of the sensor due to hydrogen injection in real time.

If there was a reaction when hydrogen was injected, it was confirmed that the voltage rapidly decreased from the voltage-time graph and then the hydrogen was stopped by adjusting MFC and it was confirmed whether the time-voltage graph returned to its original state from the oscilloscope.

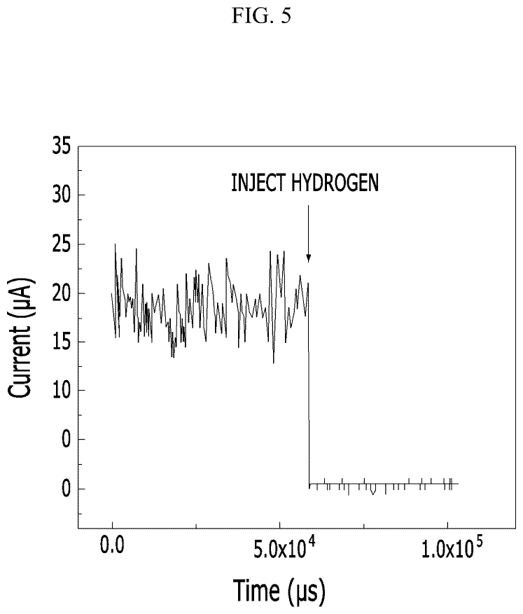

FIG. 5 is a result of measuring a reaction time when hydrogen is injected at 1% hydrogen concentration into a gas sensor according to embodiment 1-1 of the present disclosure.

Referring to FIG. 5, it was confirmed that when hydrogen was injected, ultra-fast detection characteristic of 300 μs or lower was shown. This is a reaction time caused by the limitation of the measurement equipment and an actual measurement time is expected to be ns level.

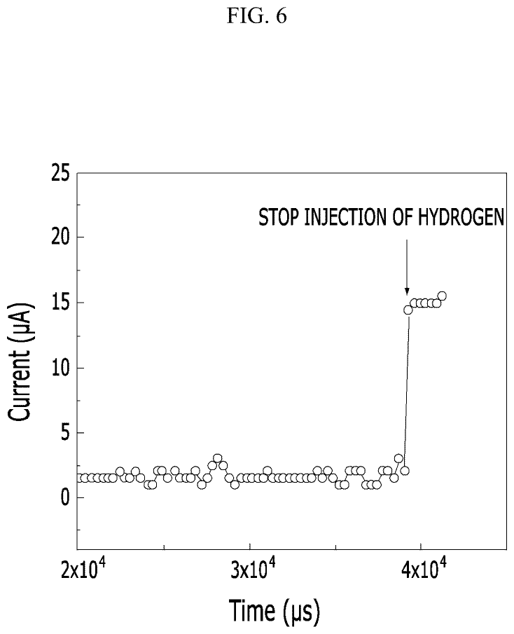

FIG. 6 is a result of measuring a recovery time when injection of hydrogen at 1% hydrogen concentration is stopped into a gas sensor according to embodiment 1-1 of the present disclosure.

Referring to FIG. 6, it was confirmed that when the hydrogen injection was stopped, rapid current rising was shown, which was very short in the nano second time range. In the related art, the recovery time of the gas sensor was several minutes.

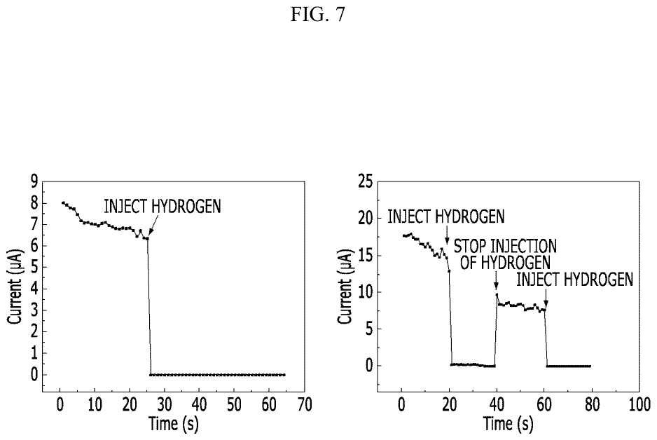

FIG. 7 is a results of measuring the reaction time and recovery time when hydrogen injection and hydrogen injection are stopped at 0.5% hydrogen concentration into the gas sensor according to embodiment 1-2 of the present disclosure.

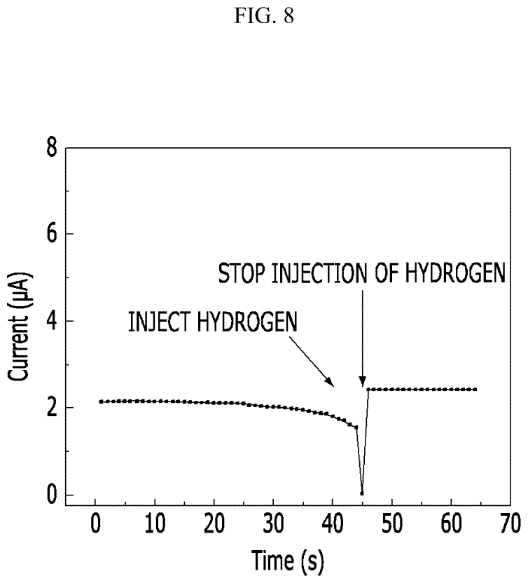

FIG. 8 is a results of measuring the reaction time and recovery time when hydrogen injection and hydrogen injection are stopped at 0.1% hydrogen concentration into the gas sensor according to embodiment 1-3 of the present disclosure.

Referring to FIGS. 6 to 8, it was confirmed that the gas sensor according to embodiment 1-3, which is a sensor with a low 2DEG electron concentration (i.e., a device with a low current level of the sensor in the standby state before hydrogen injection), is advantageous in detecting low concentration hydrogen.

The above-description of the present disclosure is illustrative only and it is understood by those skilled in the art that the present disclosure may be easily modified to another specific type without changing the technical spirit of an essential feature of the present disclosure. Thus, it is to be appreciated that embodiments described above are intended to be illustrative in every sense, and not restrictive. For example, each component which is described as a singular form may be divided to be implemented and similarly, components which are described as a divided form may be combined to be implemented.

The scope of the present disclosure is represented by the claims to be described below rather than the detailed description, and it is to be interpreted that the meaning and scope of the claims and all the changes or modified forms derived from the equivalents thereof come within the scope of the present disclosure.

1. A gas sensor, comprising:
a first oxide layer;
a second oxide layer disposed on the first oxide layer;
an inactive electrode formed at an interface of the first oxide layer and the second oxide layer; and
an active electrode disposed on the second oxide layer,
wherein the inactive electrode includes a two-dimensional electron gas (2DEG) layer, and a metal filament which connects the active electrode and the inactive electrode and is derived from the active electrode is included in the second oxide layer.

2. The gas sensor according to claim 1, wherein when gas is injected in a state in which a voltage is applied to the gas sensor, the connection of the metal filament and the inactive electrode is disconnected and when the injection of the gas is stopped, the metal filament and the inactive electrode are connected again.

3. The gas sensor according to claim 2, wherein when the gas is injected, due to the heat transmission by the gas, a temperature of a part of the metal filament adjacent to the inactive electrode exceeds a critical point so that the metal filament collapses.

4. The gas sensor according to claim 3, wherein in the collapse state, a tunnel gap which is an interval between an end portion of the metal filament and the inactive electrode is 0.1 nm or larger and 0.5 nm or smaller.

5. The gas sensor according to claim 1, wherein a diameter of a part of the metal filament adjacent to the active electrode is larger than a diameter of a part of the metal filament adjacent to the inactive electrode.

6. The gas sensor according to claim 2, wherein the gas includes one selected from the group consisting of hydrogen, ammonia, benzene, butane, heptane, hexane, pentane, propylene, isobutane, ethylene, ethane, isopentane, isobutylene, methane, propane, hydrogen sulfide, carbon monoxide, and combinations thereof.

7. The gas sensor according to claim 1, further comprising:
a buffer layer disposed between the active electrode and the second oxide layer.

8. The gas sensor according to claim 1, wherein the first oxide layer and the second oxide layer are each independently formed of oxide including a metal element selected from the group consisting of titanium (Ti), zinc (Zn), aluminum (Al), hafnium (Hf), zirconium (Zr), gallium (Ga), tungsten (W), indium (In) and combinations thereof.

9. The gas sensor according to claim 1, wherein the active electrode includes one selected from the group consisting of copper (Cu), silver (Ag), platinum (Pt), gold (Au), molybdenum (Mo), nickel (Ni), and combinations thereof.

10. A gas sensor array, comprising:
a plurality of gas sensors which is disposed in a M×N matrix along a first horizontal direction and a second horizontal direction intersecting the first horizontal direction;
a plurality of first signal lines which is disposed in the second horizontal direction to be spaced from each other and extends to the first horizontal direction to be electrically connected to active electrodes of M gas sensors disposed in one line along the first horizontal direction, among the plurality of gas sensors, and
a plurality of second signal lines which is disposed in the first horizontal direction to be spaced from each other and extends to the second horizontal direction to be electrically connected to inactive electrodes of N gas sensors disposed in one line along the second horizontal direction, among the plurality of gas sensors,
wherein the plurality of gas sensors includes the gas sensor according to any one of claims 1 to 9, and the plurality of gas sensors has different thicknesses of parts of the metal filament which are in contact with the inactive electrode.

11. The gas sensor array according to claim 10, wherein when the gas is injected, due to the heat transmission by the gas, a temperature of a part of the metal filament adjacent to the inactive electrode exceeds a critical point so that the metal filament collapses and the collapse selectively occurs according to a thickness of the metal filament and a concentration of the injected gas.

12. The gas sensor array according to claim 10, wherein each of the plurality of gas sensors further includes:
a support structure which is disposed on one corresponding second signal line, among the second signal lines and has a pillar structure,
the first oxide layer is disposed to cover a side surface and an upper surface of the support structure, and the second oxide layer is disposed to directly cover the first oxide layer on the side surface and the upper surface of the support structure.

13. The gas sensor array according to claim 12, wherein the support structure includes a pillar structure having a circular or polygonal cross-sectional shape.

14. The gas sensor array according to claim 12, wherein the second signal lines are in direct contact with cross-sectional surfaces of the inactive electrodes exposed in an area adjacent to a lower end portion of the support structure.

15. The gas sensor array according to claim 12, further comprising:
a filling layer disposed to fill a space between the plurality of gas sensors,
wherein active electrodes of the gas sensors are exposed through an upper surface of the filling layer to be electrically connected to the first signal line.

16. The gas sensor array according to claim 15, wherein the filling layer includes a first layer which fills a space between the gas sensors and a second layer formed above the first layer, the first layer exposes a part of an upper surface of the second oxide layer of the gas sensors, and the second layer includes a plurality of through-holes through which the active electrodes of the gas sensors pass.
